(19) United States
(12) Patent Application Publication
(10) Pub. No.: US 2025/0046452 A1
(43) Pub. Date: Feb. 6, 2025

(54) A METHOD FOR CANCER PROGNOSTICATION USING PERFUSION KINETIC ANALYSIS OF STANDARD-OF-CARE DCE MRIS

(71) Applicant: SIMBIOSYS, INC., Champaign, IL (US)

(72) Inventors: John Andrew COLE, Champaign, IL (US); Joseph Ryan PETERSON, Champaign, IL (US)

(21) Appl. No.: 18/717,324

(22) PCT Filed: Dec. 6, 2022
(86) PCT No.: PCT/US2022/052053

§ 371 Date: Jun. 6, 2024

Related U.S. Application Data
(60) Provisional application No. 63/286,194, filed on Dec. 6, 2021.

(51) Int. Cl. G16H 50/20; G06T 7/00

(52) U.S. Cl. CPC G16H 50/20; G06T 7/0016; G06T 2207/10096; G06T 2207/30068

(57) ABSTRACT

A method of modeling that allows for kinetic parameters to be extracted from DCE-MRIs performed with the temporal resolutions commonly used in the clinical setting is described herein. The kinetic parameters can be combined with analytic models of cancers to create predictors of disease recurrence.

### CROSS-REFERENCE TO RELATED APPLICATION

This application claims priority to U.S. provisional patent application No. 63/286,194, filed Dec. 6, 2021, which is hereby incorporated in its entirety.

### BACKGROUND

Dynamic contrast enhanced (DCE) magnetic resonance imaging (MRI) has been used in the diagnosis and treatment planning processes for cancers. Chief among its use is the identification of tumors in soft tissues, where tumor-induced morphological and perfusion-related changes result in leaky and irregular vasculature which is readily highlighted by a paramagnetic contrast agent. While DCE-MRI is commonly used for clinical diagnosis, the images contain rich information about the dynamics and extent of vascular leakiness. Using this information, the dynamics and leakiness of vasculature can be modeled as kinetic processes. Toft's model (TM) is a two-compartment model describing the transport of contrast agent from the vasculature into the extracellular-extravascular space (EES). The model accounts for two independent variables, Kₜᵣₐₙₛ and vₑ, the former representing the transport rate from vasculature into the EES, and the latter representing the volume of EES in each pixel of an image. The extended Toft's model, further accounts for vₚ, the volume of vasculature in each pixel. However, in order to fit a Toft's-like model, an assumption of a form for the temporal evolution of the vascular concentration of contrast agent is used. A so-called “arterial input function” (AIF) can be measured, by sequentially imaging small regions of interest near an artery, or one can be assumed. The Parker AIF is characterized by a pronounced narrow peak (the first passage of the bolus) followed by a second less-pronounced peak (associated with the second passage), and finally a long exponential decay. However, the early transients of the AIF are so rapid that accurate fitting of a Toft's-like model requires periods of time on the order of 10 seconds or less between images.

Disadvantageously, DCE-MRIs of the breast consist of temporal resolutions of a minute or more, and entire time-traces may be composed of a single pre-contrast image, with two and five post-contrast images. As such, high temporal resolution MRI is required to fit kinetic parameters reliably. This has limited the use of kinetic analyses of DCE MRIs to the research setting, and required the use of ultrafast MRI technologies, and/or small regions of interest.

Thus, a need exists for a method of modeling that circumvents the limitation of current models, thereby allowing kinetic parameters to be extracted from DCE-MRIs performed with the temporal resolutions commonly used in the clinical setting. A further need exists to combine the obtained kinetic parameters with analytic models of cancers to create predictors of disease recurrence.

### SUMMARY

Prognosis of cancer can be improved by way of one or more of the embodiments herein. In particular, perfusion kinetic-analysis derived from DCE-MRIs, wherein a bolus injection of contrast agent, usually gadolinium-based, is administered, and multiple images are produced, can be used when imaging breast cancer patients. It offers insight into the anatomy of a breast tumor, in addition to the associated vascularization and perfusion. However, DCE-MRIs, such as those obtained from the breast, commonly use temporal resolutions on the order of minutes and entire time-traces may be composed of a single pre-contrast image, and between two and five post-contrast images. The model disclosed herein improves the time-limitation of traditional Toft's-like modeling and can be used to determine diagnosis, prognosis, and tailor treatment of disease on an individual basis

Accordingly, a first example embodiment involves a computer-implemented method comprising obtaining a DCE-MRI for a patient. The first example embodiment may also involve defining a form of a pharmacokinetic model and fitting the parameters of the pharmacokinetic model to the DCE-MRI using an optimization method. The first example embodiment may also generate, by using the fitted parameters of the optimization method, an individualized prognosis of disease recurrence for a patient.

In a second example embodiment, an article of manufacture may include a non-transitory computer-readable medium, having stored thereon program instructions that, upon execution by a computing system, cause the computing system to perform operations in accordance with the first and/or second example embodiment.

In a third example embodiment, a computing system may include at least one processor, as well as memory and program instructions. The program instructions may be stored in the memory, and upon execution by the at least one processor, cause the computing system to perform operations in accordance with the first and/or second example embodiment.

In a fifth example embodiment, a system may include various means for carrying out each of the operations of the first and/or second example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF DRAWINGS

FIG. 1 illustrates a schematic drawing of a computing device, in accordance with example embodiments.

FIG. 2 illustrates a schematic drawing of a server device cluster, in accordance with example embodiments.

FIG. 3 depicts kinetic parameter maps constructed using MRIs.

FIG. 4 depicts a representative set of covariates of a particular embodiment of a prognostic method incorporating kinetic parameters KP1 and KP2 into prediction of recurrence free survival in breast cancer patients. Part A depicts a cohort of 334 patients treated with neoadjuvant chemotherapy (COHORT 1). Part B depicts a cohort of 149 patients treated with neoadjuvant chemotherapy (COHORT 2).

FIG. 5 depicts the capability of model outputs in predicting recurrence free survival in adjuvant and neoadjuvant treated breast cancer patients. Part A depicts patients treated with chemotherapy and/or anti-HER2 therapy. Part B depicts patients treated with only chemotherapy.

FIG. 6 depicts the capability of model outputs in predicting recurrence free survival in neoadjuvant chemotherapy treated breast cancer patients. Part A depicts patients that had a pathological complete response with treatment. Part B depicts patients that had residual disease after treatment.

FIG. 7 depicts the capability of model outputs in predicting recurrence free survival in neoadjuvant chemotherapy treated breast cancer patients. Part A depicts patients that had a pathological complete response with treatment. Part B depicts patients that had residual disease after treatment.

FIG. 8 depicts the capability of model outputs in discriminating recurrence free survival for patients with low or mid OncotypeDx® score). Part A depicts patients with low or mid OncotypeDx Score. Part B depicts patients with mid OncotypeDx Score.

FIG. 9 depicts flow charts, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized, and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

The examples described below are generally related to human metabolic systems and their influence on the progression of various types of cancers. Nonetheless, the approach described could be used for non-human subjects and/or for diseases or conditions other than cancer.

Further, the terms “individual”, “patient”, and “subject” may be used interchangeably herein unless context suggests otherwise.

### I. Example Computing Devices and Cloud-Based Computing Environments

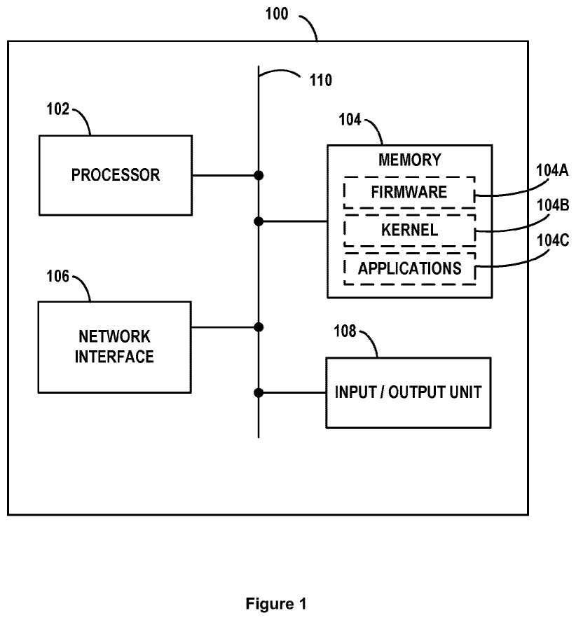

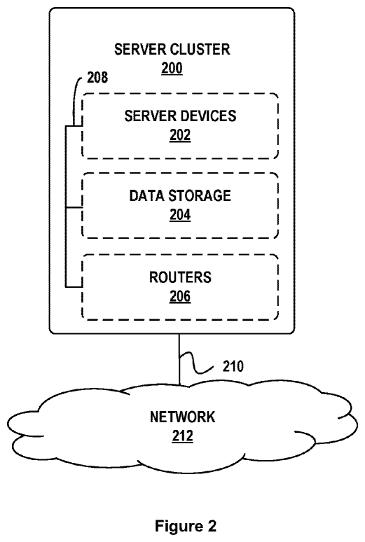

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently used instructions and data.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling, and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications. Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally, or alternatively, computing device 100 may communicate with other devices using a universal serial bus (USB) or high-definition multimedia interface (HDMI) port interface, for example.

In some embodiments, one or more computing devices like computing device 100 may be deployed to facilitate computation and use of metabolic models. The exact physical location, connectivity, and configuration of these computing devices may be unknown and/or unimportant to client devices. Accordingly, the computing devices may be referred to as “cloud-based” devices that may be housed at various remote data center locations.

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid-state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as the hypertext markup language (HTML), the extensible markup language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively, or additionally, JAVA® may be used to facilitate generation of web pages and/or to provide web application functionality.

### II. Clinically Relevant Pharmacokinetic Modelling Framework for Prognosis

Standard clinical DCE-MRIs contain relatively few, perhaps as few as two, post-contrast images. To avoid pixel-wise overfitting of pharmacokinetic models, the number of free parameters in the model does not usually exceed the number of post-contrast images. Further, standard clinical DCE-MRIs have coarse temporal resolution, such that they are unable to capture early transients in images. A fitting strategy using post-contrast images overcomes or compensates for these constraints. The fitting strategy is advantageous because later post-contrast images tend to be of lower signal intensity than images captured during the initial bolus. As such, they tend to exhibit a more linear relationship between contrast agent concentration and signal intensity, obviating the need for the flip-angle MRI sequences that would otherwise be necessary to relate contrast agent concentration and signal intensity. However, nonlinear fits are intrinsically difficult but a pharmacokinetic model that allows for a readily accessible “initial guess” that is likely close to the final fitting parameter values improves the process.

In a first embodiment of the method disclosed herein utilizes fitting, via linear or nonlinear optimization methods, a pharmacokinetic model to as few as three timepoints from the DCE-MRI. The first embodiment may involve the use of global parameter constraints, such as those that may be extracted from the Parker AIF or other sources, during the fitting process. During the fitting process, a segmentation of the tissues within the region of interest that includes, but is not limited to, regions of tumor, vasculature, and other healthy tissues may be utilized. The first embodiment may also involve generating, by including parameters from the fitted pharmacokinetic model with other clinical and demographic parameters, an individualized prognosis of disease recurrence for a patient. The model parameters can be combined with clinical and demographic features, for example age, race, cancer histological properties. The parameters can be combined with statistical or machine learning approaches to create a personalized prognosis for a patient or a population of patients

A second example embodiment utilizes obtaining a plurality of DCE-MRIs for a set of patients. The second embodiment can define a form of a pharmacokinetic model, and may involve fitting, via linear or nonlinear optimization methods, the pharmacokinetic model to as few as three timepoints from each of the DCE-MRI for patients.

Global parameter constraints, such as those extracted from the Parker AIF or other sources, can also be utilized during the fitting process. Tissue segmentation within the region of interest, including but not limited to tumor, vasculature, and other healthy tissues can also be incorporated into the second embodiment. The second embodiment may also include parameters from the fitted pharmacokinetic model with other clinical and demographic parameters to generate a prognostic model of disease recurrence for a population of patients. Individual prognosis of disease recurrence based on the population model of disease recurrence can also be a component of the second embodiment.

### III. Example Pharmacokinetic Model

In an example embodiment, the pharmacokinetic model consists of a formulation wherein the concentration of contrast agent (e.g., a radioactive tracer or some other substance) in a voxel, c(t), is given as the sum of vascular and extra-vascular (or tissue) concentrations:

$$c ( t ) = φ cv ( t ) + ct ( t ) \qquad ( 1 )$$

where φ represents the volume-fraction of vasculature within the voxel, cv(t) represents the vascular concentration, and ct(t) represents the tissue concentration of the agent. The time-derivative of this equation (1) immediately yields:

$$dc ( t ) / dt = φ dcv ( t ) / dt + dct ( t ) / dt \qquad ( 2 )$$

In the late post-contrast limit, the vascular concentration of the agent decays exponentially. As such, in some embodiments, the vascular concentration and time derivative can be given by:

$$cv ( t ) = cv 0 exp [ - γ t ] , and \qquad ( 3 )$$

$$dcv ( t ) / dt = - γ cv 0 exp [ - γ t ] , \qquad ( 4 )$$

where γ is the decay constant, and cv0 is the vascular concentration at the first post-contrast timepoint within the voxel.

In some embodiments, ct(t) will be assumed to only depend on the concentration within a pixel, and changes in time through the transport of contrast agent in and out of the local vasculature. The transport out of the local vasculature is proportional to the vascular density, φ, the vascular agent concentration, cv(t), and some measure of the “leakiness” of the vasculature, k. The transport into the vasculature will be proportional to φ and k, but it can also be proportional to the concentration of the agent in the fraction of tissue that is not vasculature, i.e., ct(t)/(1−φ). We can make this explicit as:

$$dct ( t ) / dt = φ k cv ( t ) - φ k ct ( t ) / ( 1 - φ ) \qquad ( 5 )$$

In some embodiments, equation (1) can be solved for ct(t), and the result inserted into the right-hand side of equation (5) to give the time rate of change equation:

$$dct ( t ) / dt = φ k cv ( t ) = φ k [ c ( t ) - φ cv ( t ) ] / ( 1 - φ ) \qquad ( 6 )$$

which can be used to reformulate equation (2) as:

$$dc ( t ) / dt = - φ γ cv 0 exp [ - γ t ] + φ k cv 0 exp [ - γ t ] - φ k [ c ( t ) - φ cv 0 exp [ - γ t ] ] / ( 1 - φ ) \qquad ( 7 )$$

This has the explicit solution:

$$c ( t ) = A exp [ - γ t ] + ( c 0 - A ) exp [ - KP 2 t ] \qquad ( 8 )$$

where KP2=φ k/(1−φ), c0 represents the measured contrast agent concentration in the first post-contrast image (provided it is late enough to ensure the AIF is in the exponential decay regime), and A=cv0 φ (k+KP2−γ)/(KP2−γ).

In some embodiments of the invention, the parameter cv0 can be measured directly from regions of the first late post-contrast image that are known to represent blood vessels.

In some embodiments cv0 may be treated as a variable that can be fit.

In some embodiments, cv0 may be treated as a fitted parameter, but it may be constrained to remain within some specified range of values or biased toward values similar to that which may be measured within regions known to represent blood vessels.

In some embodiments, the parameter γ can also be directly measured using regions known to represent blood vessels, for example by fitting an exponential decay across multiple late post-contrast images or by performing some other similar analysis.

In some embodiments, values for γ may be taken from publications, such as that which appears in Parker G, Roberts C, Macdonald A, et al. (2006) Experimentally derived functional form for a population-averaged high-temporal-resolution arterial input function for dynamic contrast-enhanced MRI. Magn Reson in Medicine 56:993-1000. In such embodiments, all that remains is a voxel-by-voxel fit of Eq. 8 to the measured signal intensities by varying φ and/or k.

In some embodiments both φ and k are treated as fitted parameters.

In some embodiments φ might be fitted while k might be held fixed at some reference value, possibly taken from the literature.

In still other embodiments, φ might be held fixed at some reference value while k is treated as a fitted parameter.

In some embodiments, the fitting procedure is performed using a non-linear optimization software package.

In some embodiments, the fitting procedure may use machine learning methods such as automatic differentiation and optimization.

In some embodiments, the post-contrast images may be used to define an initial guess for the parameters φ, k, or both.

### IV. Experimental Results

Derived parameters may be defined as KP1=φ k and KP2=φ k/(1−φ), as noted herein the remainder of this document.

### A. Model and DCE-MRI

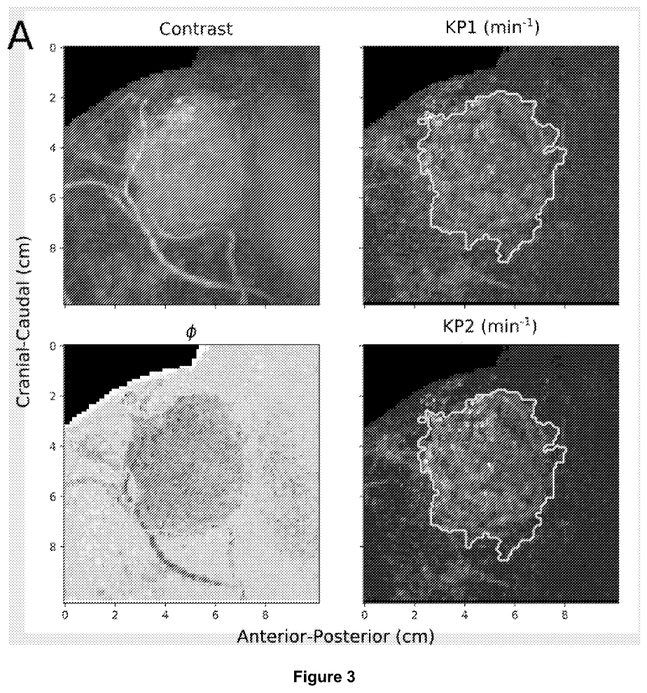

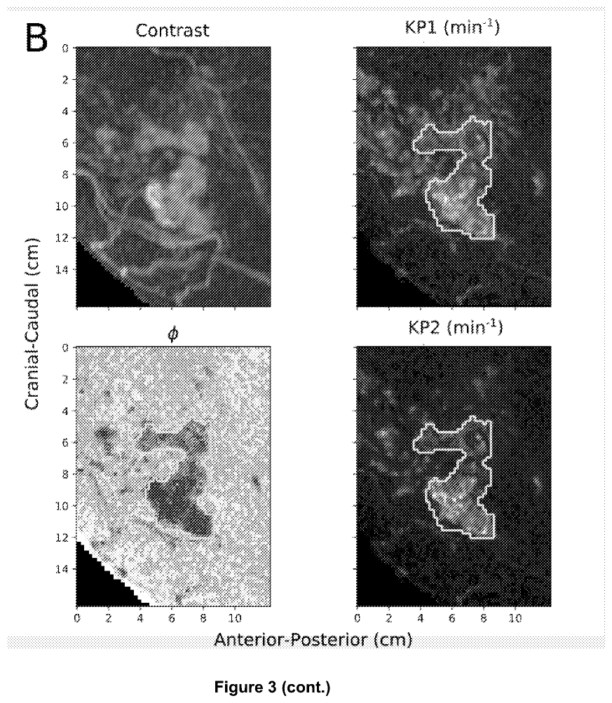

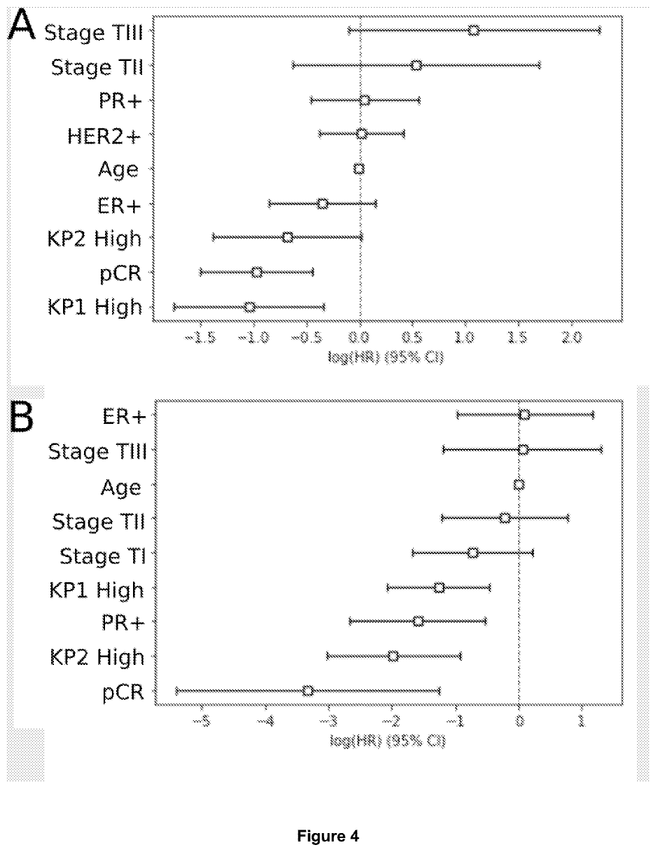

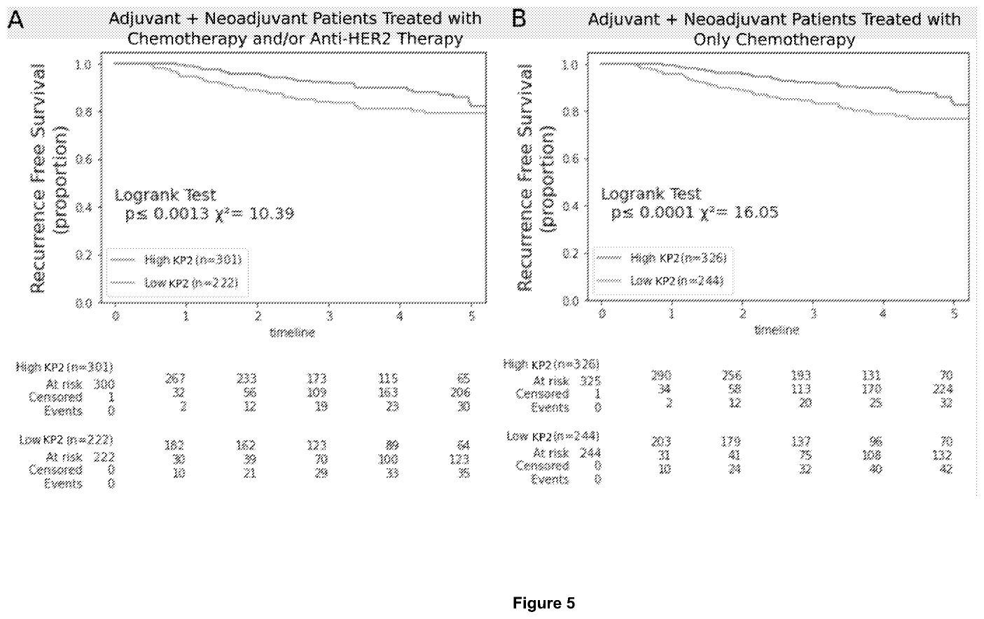

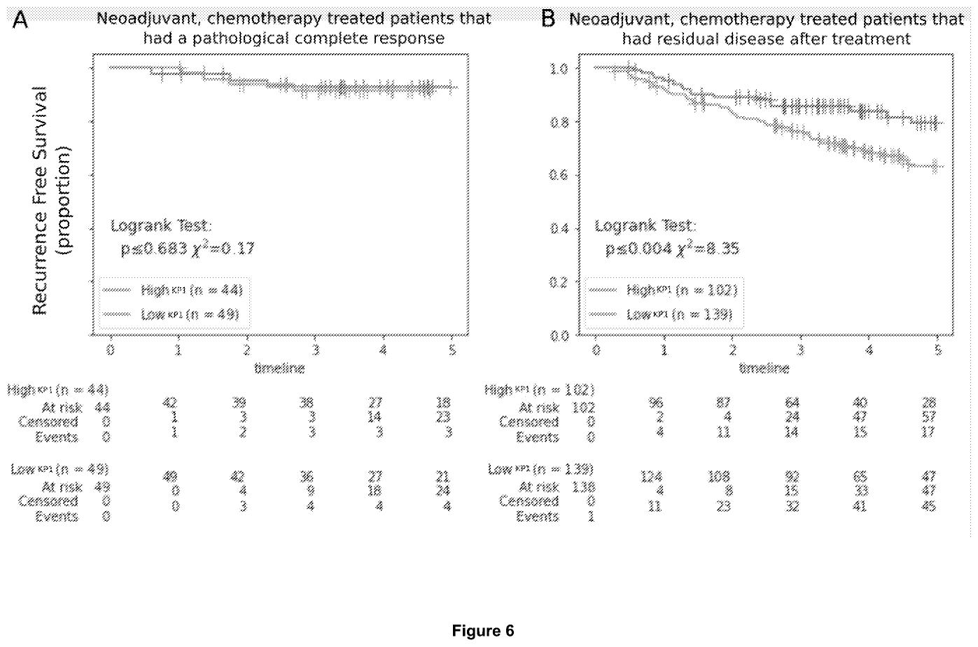

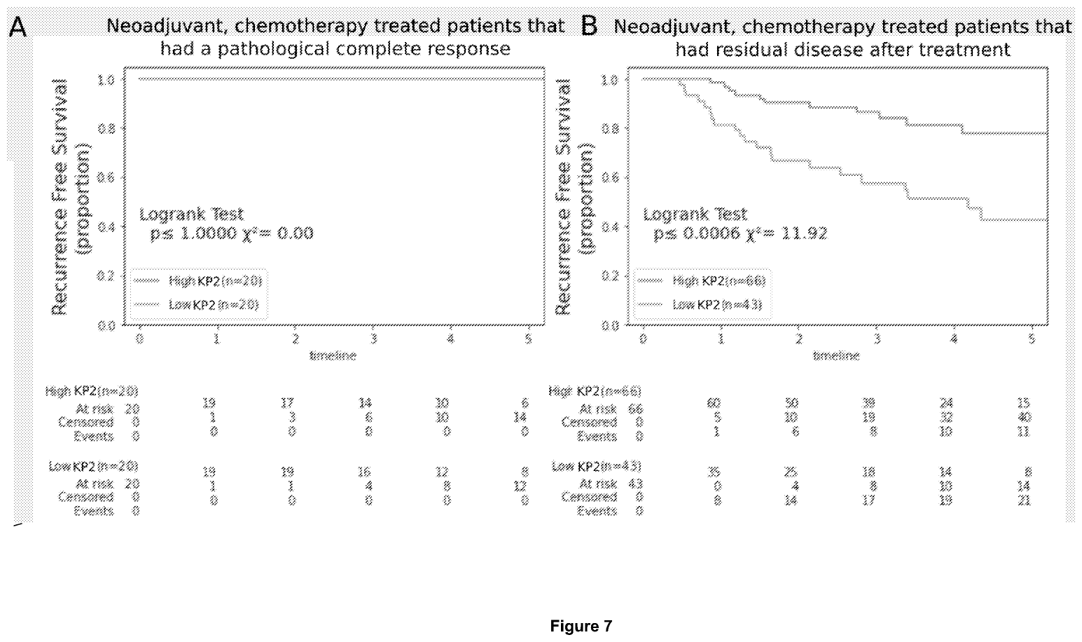

A model and DCE-MRI can be used with a fitting procedure to extract three dimensional maps of KP1 and KP2. FIG. 3 depicts representative images of these parameter maps from two breast cancer patients. Subsequent post-processing is used to extract median KP1 and KP2 parameter values from a delineated tumor region for each patient. The model was used on two independent cohorts of early-stage breast cancer patients treated with neoadjuvant chemotherapy (334 patients in COHORT 1, and 149 patients in COHORT 2; FIG. 4). Only Pre-treatment DCE-MRIs were used in all cases. Median intra-tumoral KP1 and KP2 values were pooled across cohorts, and a median of medians for each kinetic parameter determined and used to categorize each patient as high- or low-KP1 and high- or low-KP2. A subsequent Cox proportional hazard analysis showed that both high-KP1 and high-KP2 status were associated with significantly decreased risk of disease recurrence. Hazard ratios associated with high-KP1 and high-KP2 status were comparable to a pathologically complete response to neoadjuvant chemotherapy (see FIG. 4). Significant differences in recurrence free survival between high vs. low KP2 status patients was independent of the use of HER2-targeted therapies like trastuzumab and/or pertuzumab (see FIG. 5), and high vs. low KP1 status was able to stratify risk of recurrence among patients with residual disease following neoadjuvant chemotherapy (see FIGS. 6 & 7). No significant differences were seen among patients who achieved pathological complete response.

### B. KP1/KP2

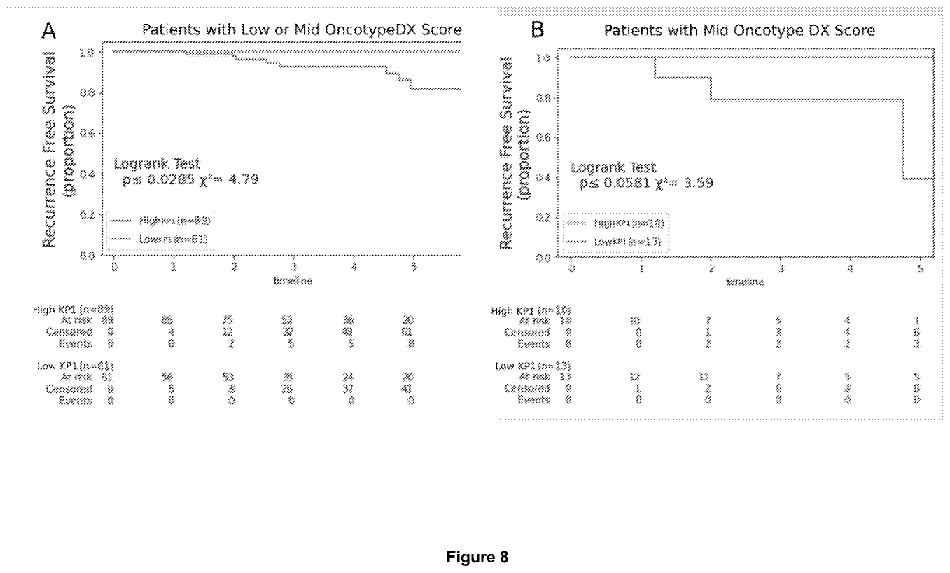

Kinetic parameters KP1 or KP2 can be used in the interpretation of OncotypeDx® scores. Patients with higher scores tend to be more responsive to chemotherapy, and also more likely to experience disease recurrence, while patients with lower scores have a lower risk or recurrence and also tend to respond less to chemotherapy. In practice this is often used to determine whether the risk of administering chemotherapy outweighs the potential clinical benefit. The challenge occurs among patients with scores in the middle of the range, for which these risks and benefits are less well defined. A subset of hormone-receptor positive patients in COHORT 2 had low or mid OncotypeDx scores with low KP1 scores being associated with significantly longer recurrence free survival (see FIG. 8).

### V. Prognostic Utility

The current disclosure demonstrates technical advantages related to cancer prognostication and risk assessment. Currently, a number of markers are used to determine recurrence risk profiles and treatment options but require the use of specialized laboratory tests and trained personnel.

The current disclosure is advantageous in that it provides a practical method for extracting actionable prognostic information directly from standard-of-care DCE MRIs. This provides significant advantages over traditional marker-based recurrence risk assessments that are time consuming, are dependent upon the skill level of trained personnel, and expensive equipment and testing methods.

Further, physicians can use results of the current disclosure to select between more or less aggressive therapeutic options, for example selecting more aggressive chemotherapy regimens for patients with worse prognosis, or forgoing chemotherapy among patients with especially good prognosis (e.g., mid, or low OncotypeDx score, with low KP1 status). Physicians may also opt for more or less frequent post treatment monitoring, perhaps seeing patients with better prognosis less frequently than those with worse. Through these and other mechanisms patient care can be personalized, ultimately leading to better outcomes for more patients.

### VI. Example Operations

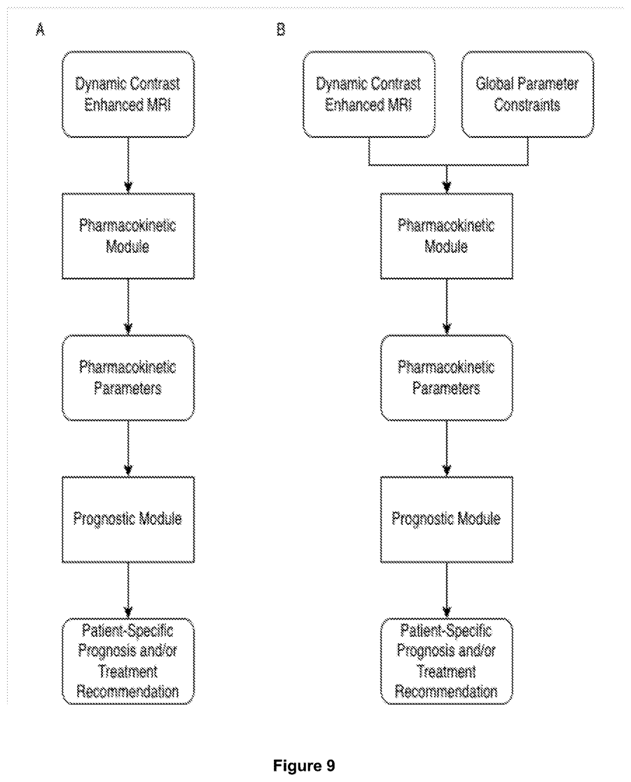

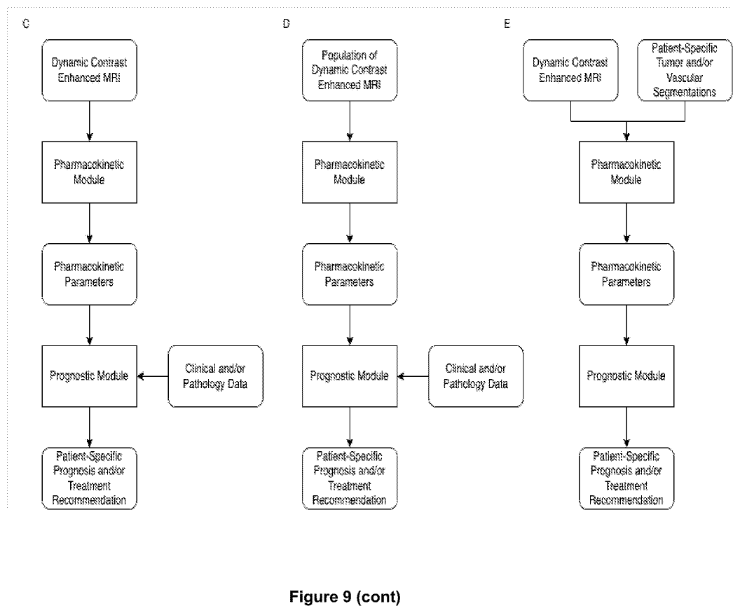

FIG. 9 demonstrates the flow of information through possible embodiments of the invention. In one embodiment (Part A), patient DCE-MRI data is used as input, the microvascular model is used to extract KP1 and KP2 maps, a prognostic module is used to determine prognostic scores from the KP1 & KP2 maps. For example, statistical measures including intratumoral median KP1 and KP2 can be computed and compared to a reference value to determine high or low status, and a prognosis and/or treatment recommendation is returned.

In another embodiment (Part B), additional parameter constraints are input to the microvascular model. These may be ranges of possible values for φ, k, cv0, and/or γ, reference values toward which they may be biased, or values to which they may be fixed.

In another embodiment, other statistical measures such as mean, standard deviation, quantiles, or histograms of KP1 or KP2 might be computed and used to determine prognosis.

In another embodiment (Part C), the prognostic module may take inputs other than simply those produced by the microvascular model. These may include, but are not limited to, molecular, histological, or other pathological markers or scores (e.g., hormone receptor or Her2 statuses, OncotypeDx scores, tumor grade, tumor stage, etc.), demographic data (e.g., age, ethnicity, etc.), or medical history data (e.g., previous diseases, comorbidities, etc.).

In another embodiment (Part D), multiple prognostic modules may be used that produce different prognoses or recommendations for treatment options or are used to perform more complex prognostic analyses.

In another embodiment (Part E), image segmentations that specify regions of different tissue types (e.g., blood vessels, tumor, etc.) serve as inputs for the microvascular model. The segmentations aid in estimation or fitting of parameters such as cv0, and/or γ.

In still another embodiment, the prognostic module utilizes kinetic parameters and other clinical and/or pathological data, from a population of patients, and produces population level prognostic and/or treatment decision outputs.

### VII. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more, or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively, or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as non-transitory computer readable media that store data for short periods of time like register memory and processor cache. The non-transitory computer readable media can further include non-transitory computer readable media that store program code and/or data for longer periods of time. Thus, the non-transitory computer readable media may include secondary or persistent long-term storage, like ROM, optical or magnetic disks, solid-state drives, or compact disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures. While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
obtaining dynamic contrast enhanced (DCE) magnetic resonance imaging (MRI) for a patient;
defining a form of a pharmacokinetic model in which vascular concentration of a contrast agent introduced to the patient decays exponentially during at least three timepoints of the DCE MRI;
fitting parameters of the pharmacokinetic model to the DCE MRI using an optimization method; and
generating, by using the parameters, an individualized prognosis of disease recurrence for the patient.

2. The computer-implemented method of claim 1, wherein the DCE MRI is of a breast region of the patient.

3. The computer-implemented method of claim 1, wherein the three timepoints occur after introduction of the contrast agent.

4. The computer-implemented method of claim 1, wherein the optimization method comprises linear optimization.

5. The computer-implemented method of claim 1, wherein the optimization method comprises nonlinear optimization.

6. The computer-implemented method of claim 1, wherein the optimization method is comprises a machine learning technique.

7. The computer-implemented method of claim 6, wherein the machine learning technique comprises automatic differentiation and optimization.

8. The computer-implemented method of claim 1, wherein at least some of the parameters are constrained during fitting.

9. The computer-implemented method of claim 1, wherein the individualized prognosis is performed by a statistical or machine learning model.

10. The computer-implemented method of claim 9, wherein the statistical or machine learning model comprises one or more of: linear regression, logistic regression, support vector machines, Cox Proportional Hazards regression, or deep learning.

11. The computer-implemented method of claim 1, wherein clinical, demographic, or cancer information are used in conjunction with the parameters to generate the individualized prognosis.

12. The computer-implemented method of claim 1, wherein an initial value of the vascular concentration of the contrast agent (cv0) is measured directly from regions of the DCE MRI specified in a first post-contrast image known to be blood vessels.

13. The computer-implemented method of claim 12, wherein the vascular concentration is one of the parameters.

14. The computer-implemented method of claim 13, wherein the vascular concentration is constrained to remain within a specified range of values.

15. The computer-implemented method of claim 12, wherein the vascular concentration is biased toward values measured within regions known to be blood vessels.

16. The computer-implemented method of claim 1 wherein a decay constant (γ) of the pharmacokinetic model is determined using regions of the DCE MRI known to represent blood vessels.

17. The computer-implemented method of claim 16, wherein the decay constant is determined by fitting an exponential decay across multiple post-contrast images of the DCE MRI.

18. The computer-implemented method of claim 16, wherein the decay constant is determined based on measurements from a population of patients.

19. The computer-implemented method of claim 1, wherein the parameters include vascular density (φ) and leakiness of vasculature (k).

20. The computer-implemented method of claim 19, wherein the vascular density is fitted while the leakiness of vasculature is fixed at a reference value.

21. The computer-implemented method of claim 19, wherein the vascular density is fixed at a reference value and leakiness of vasculature is fitted.

22. An article of manufacture including a non-transitory computer-readable medium, having stored thereon program instructions that, upon execution by a computing system, cause the computing system to perform the operations of one or more of claims 1-21.

23. A computing system comprising:
one or more processors;
memory; and
program instructions, stored in the memory, when executed by the one or more processors cause the computing system to perform the operations of one or more of claims 1-21.
